(19) United States
(12) Patent Application Publication
(10) Pub. No.: US 2005/0043658 A1
(43) Pub. Date: Feb. 24, 2005

(54) Heated wound dressing

(76) Inventor: John Robert Rix, St. Andrew Place (GB)

Correspondence Address: MCKEE, VOORHEES & SEASE, P.L.C., 801 GRAND AVENUE, SUITE 3200, DES MOINES, IA, 50309-2721, US

(21) Appl. No.: 10/494,852

(22) PCT Filed: Nov. 1, 2002
(86) PCT No.: PCT/GB02/04963

(30) Foreign Application Priority Data
Nov. 6, 2001 (GB) 0126614.7

(51) Int. Cl. A61F 5/00

(52) U.S. Cl. 602/2

(57) ABSTRACT

This invention relates to a heated wound dressing in a variety of different forms but fundamentally including an electro-conductive textile heating element which is disposed either directly in contact with or adjacent the skin of a patient, specifically over a wound inflicted on that skin.

### BACKGROUND

The temperature of wounds (typically 25-32° C.) is generally lower than that of Body Core (usually 37-38° C.). The properties of an ‘Ideal’ Wound Dressing include Thermal considerations as follows.
Systematic hypothermia can trigger thermo-regulatory vasoconstriction, which can decrease Oxygen flow to the wound. This decreased oxygen can impair Collagen deposition and oxidative killing off pathogens and can lead to reduced Healing Rate, Infection and prolonged hospitalisation.
It is well known that increasing temperature closer to Body Core temperature provides benefits to Wound healing in terms of Quality and Rate Healing. This is thought to be achieved by dilation of Arterial blood vessels, increased blood flow, increased oxygen delivery and oxidative kill off bacteria.
Temperatures significantly above Body Core temperature can cause problems with the wound/skin (e.g. Dehydration, Maceration, Burns etc.)
Some difficult to heal wounds (e.g. Venous Ulcers) which are normally treated by pressure, may also benefit from increased temperature.
Wounds can occur in many places and Dressings need to conform to the contours of the Wound Healing site.
Some systems have been proposed to provide heat to wounds by using conductive heating etched metal foil in a ‘Greenhouse’ system where the heat source is separated from the wound/skin surface. In this regard, reference should be had to the various patents and applications filed in the name of Augustine Medical Inc., including U.S. Pat. No. 6,235,047, U.S. Pat. No. 6,254,557, WO01/49233, U.S. Pat. No. 6,213,965, U.S. Pat. No. 6,080,189, U.S. Pat. No. 6,071,304 and WO99/51173.

This type of system is:
Difficult to apply over large areas
Difficult to apply to wounds where ‘flexibility’ is required
Difficult for ‘Perambulatory’ patients
Needs care to avoid direct contact of the heater with the wound/skin
Not able to be used under pressure.

The present invention proposes a system to provide advantages for Wound Healing and Patient Care whilst overcoming the inherent disadvantages of other systems. It can also be used for other types of Thermal Therapy.

Additional prior art documents to which reference should be made in assessing the current state of the art of heated wound dressings include EP0099758, WO98/46178, WO98/31311, WO93/19706, WO90/09025, WO86/05971, DE3637978 and DE3434292.

According to the present invention there is provided a heated wound dressing consisting of an electro-conductive textile and control circuitry for controlling said electroconductive textile, characterised in that the textile is place directly in contact with the wound to be treated.

According to the present invention there is provided a heated wound dressing consisting of an electro-conductive textile and control circuitry for controlling said electroconductive textile, characterised in that the textile is sealingly encapsulated within an envelope type package.

Preferably the textile is fibre, yarn or other fabric which is carbonised to enable use as a resistance heater element.

Element can be yarn, woven, knitted or non-woven-this results in Very regular, uniform, even construction and electrical resistance.

The element is also thus Soft, flexible, conformable, portable and transportable, has Good physical properties for wear resistance.

The Element is preferably provided with electrical terminals and provided with Low voltage from a suitable source

Advantageously, the resistance of element produces heating effect over whole surface area to which the element is directly applied.

Special electronic/electrical control and software may be used to control the element temperature, and such may control rate of temperature rise, maximum temperature, heating ‘on-off’ cycle and other criteria, which can be important in the healing process.

Element construction and control circuitry may ensure even temperature over the whole element surface area, and this temperature is unaffected by minor creasing or damage

Other features:

Element can be used alone in ‘Pad’ or ‘Bandage’ form

Additionally, the textile nature of the dressing enables the passing of gaseous products over the wound area and being able to do that by virtue of the woven construction of the heater element.
Energy supply can be battery or via mains isolating transformer
Heating element/system can be disposable or re-usable
Low energy requirement
Safe with built-in ‘fails safe’ protection
Can be used over dressing of choice or can be integrated with combined dressing
Can incorporate an ‘Insulating’ layer to reduce energy requirements and ensure significant heat is not lost to atmosphere

The control circuitry may rely on a feedback temperature measurment effected by a thermistor, and/or the control circuitry may operate on a thermostatic basis.

Preferably the control circuitry includes switching components and/or software and/or control to to ensure that the element is only used over a “safe” life span. This could be limited to a number of hours over a number of switchings. This could be achieved by a very small unobtrusive chip programmed to perform the ‘action of use’ counter and ‘off switch’, the final action of which would be to prevent itself from and the system from functioning further until a new pad is fitted to the system.

Non-adherent dressings have been developed to reduce trauma on dressing removal. Both traditional and non-adherent dressings have not been fully successful with many wound types in that the healing of these wounds is still under dry conditions with consequential scab formation in healing. In order to eliminate this problem, the non-adherent dressing would be used with such materials as hydrocolloids, hydrogels, transparent films and the like as appropriate.

Ideally the control circuitry would be designed to incorporate the following features:
Multi channel operation, probably 5 in total to correspond with anatomic areas.
Precise temperature control for prolonged or indefinite periods of time.
Virtually no operational temperature hysterisis.
Reciprocal temperature feedback from the patient's skin tissue.
Over temperature failsafe cut off.
Re-programmability of operational parameters.
Low energy requirements.

The software for controlling the operation of the control circuitry (i.e. front end software for initially programming the solid state devices on the control circuitry) may include
Ability to set target temperature for 5 individual dressings.
Ability to display such temperatures in real time
Ability to change such temperatures.
To have feedback from the control module that system function continued.
To be warned if system or power failure had taken place.
To re-initiate system operation without the need to re-boot.
Access a menu of pre-set symmetrical temperature and load a chosen pre-set to control module.
To receive confirmation that the pre-set had been accepted.
To have new pre-set settings automatically updated on display.

In the case where the electro-conductive textile is to be encapsulated, the following options may be suitable depending on circumstances:
Breathable and waterproof “Throw away” system where element is an integral part of the dressing;
Breathable and waterproof “Throw away” element where element is separate to the dressing;
Breathable porous “Throw away” system where element is an integral part of the dressing;
Breathable porous “Throw away” element where element is separate to the dressing;
Impermeable “Throw away” system where element is an integral part of the dressing;
Impermeable “Throw away” element where element is separate to the dressing; It was noted that where the element was encapsulated within an impermeable laminate to both sides of the element there would be no need in some cases to separately encapsulate the Buz-Bars.

Some general considerations for wound dressings may also be taken into consideration, such as:
Unsealed and with “forced” airflow over the wound area;
Unsealed but without air flow over wound area.
“Sealed” and with minimal air entry to wound area.

In a particularly preferred embodiment, the electro-conductive textile may be laminated to a dressing material.

Alternatively, the electro-conductive textile element may be unlaminated and separate from the dressing material. After use, both heating element and dressing may be thrown away. Other options are as follows:
heating element laminated but breathable (unsealed) and separate to the dressing material. Both heating element and dressing to be one use throw away system;
heating element sealed into an envelope that could be laundered or sterilised (breathable or non-breathable) and separate to the dressing material. The dressing would be throw away but the element could be removed from the envelope and the envelope discarded or sterilised.
heating element sealed into a washable/sterilisable envelope (breathable or non-breathable) and separate to the dressing material The dressing would be throw away but the element in its sealed envelope would be sterilisable/cleanable.
heating element sealed into an impermeable washable/sterilisable laminate that would be separate to the underlying dressing and would have the thermally insulating material laminated directly to its upper surface and/or as a separate layer positioned above the heating element.
Gorix E-CT heating element sealed into an impermeable laminate that would be an integral part of the underlying dressing and would have the thermally insulating material laminated directly to its upper surface and/or as a separate layer positioned above the heating element. The heating element would be a throw away item.

Most preferably, the wound dressing is provided in an initially sealed and sterilised package from which it is removed immediately prior to use, said dressing further having a release paper adhered to the surface which in use is directly applied to the wound, such that on removing the release paper from the dressing, the dressing is adhered directly to the skin.

Preferably the electro-conductive textile heating element has laminated thereto a neoprene lamination to each face of the element. The Lower face (interfacing the dressing and the subject) is in a very thin 1 mm layer of thermally conductive neoprene. The upper surface is in a very thermally efficient 3 mm neoprene with a silver Titanium reflective backing to it.

A specific embodiment of the invention is provided by way of example with reference to the accompanying drawings wherein

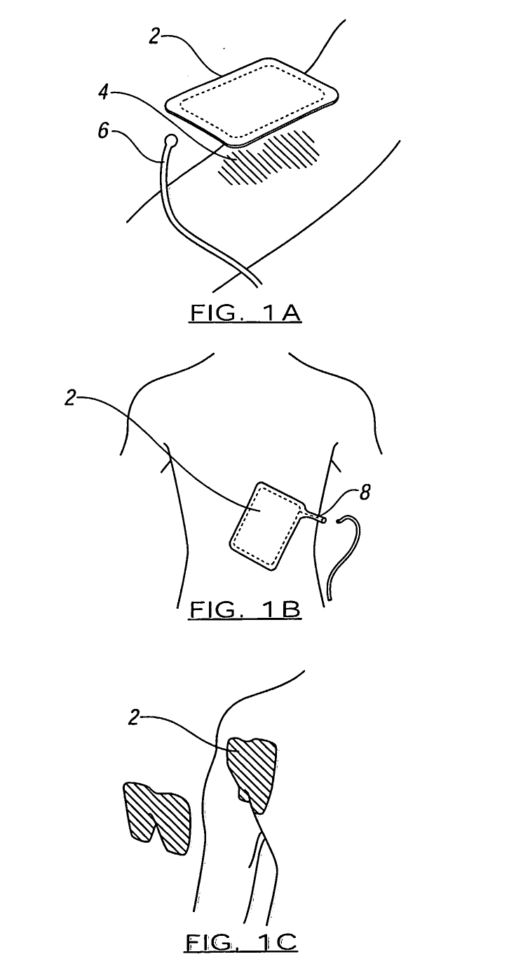

FIGS. 1A, B, C show schematically different methods of application of a dressing according to the invention,

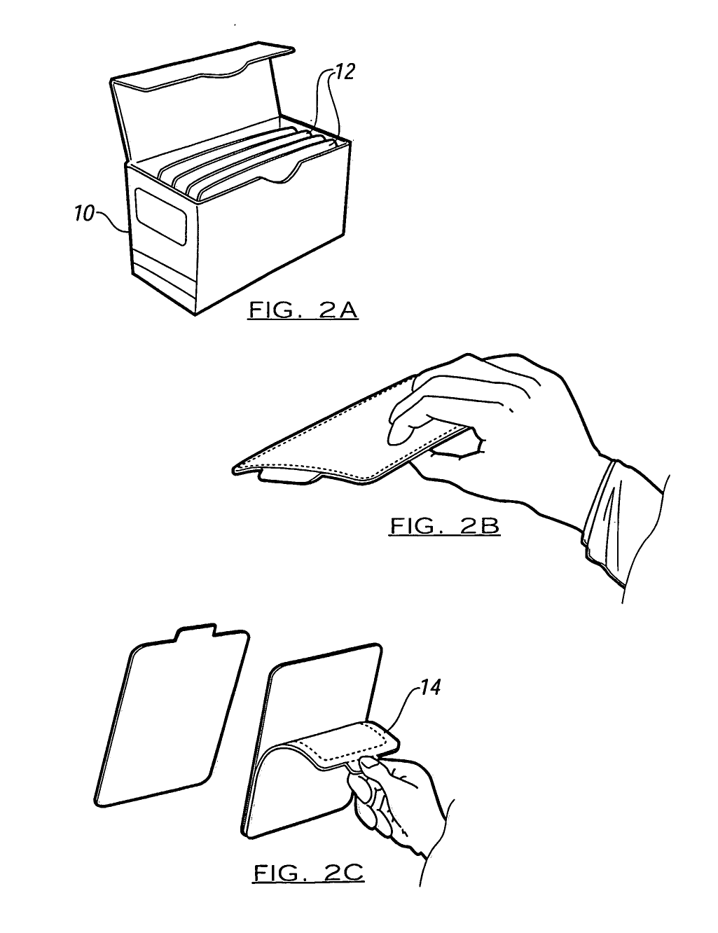

FIGS. 2A, B, C schematically demonstrate how the dressing of FIGS. 1A-C may be packaged and prepared for application to a wound, and

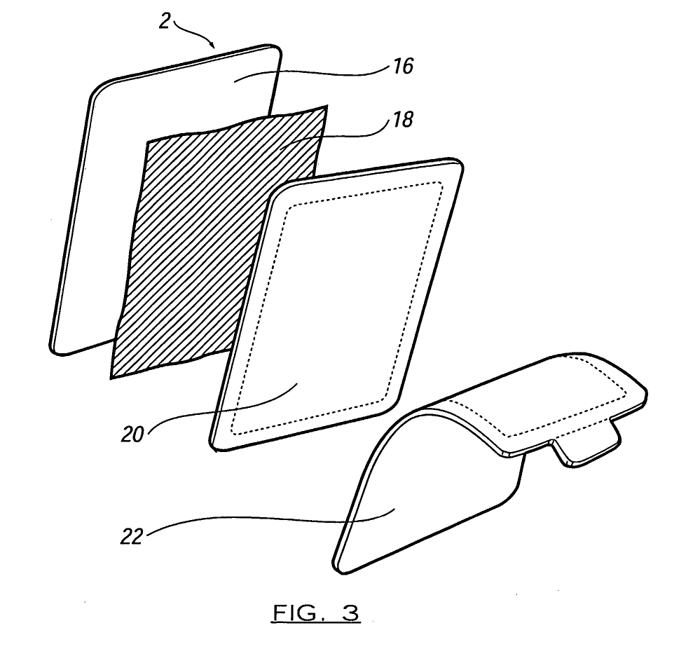

FIG. 3 shows a perspective view of the various layers which might constitute a dressing according to certain aspects or embodiments of the invention.

Briefly, FIG. 1A demonstrates how a dressing may be placed directly onto a wound 4 and connected to the power source and control system.

FIG. 1B demonstrates how an extended connection 8 may be provided so that when the dressings are placed in certain areas, they do not cause discomfort to the patient. Connection points and their location are important for both comfort and access.

FIG. 1C demonstrates how various shapes are possible using different configurations of electro-conductive textile to allow specific body parts to be treated successfully.

FIG. 2A shows how the basic dressing may be packaged in a box 10 in individual, sealed envelopes 12 to avoid contamination of each dressing and keep them clean and fresh prior to use.

FIG. 2B indicates how the dressing would be ready to use immediately on being removed from the envelopes similarly to any other ordinary sticking plaster or bandage. The dressings may come in a range of sizes suitable for the wound to be treated.

FIG. 2C demonstrates how a simple peel-off backing, such as a release paper would render the dressing ready for application to the wounded area. These simple steps make such a dressing easy to use by both patients and carers.

Finally, FIG. 3 shows how a particular dressing may be constituted, and for example the dressing 2 may be of laminated construction and comprise a breathable outer cover 16, an electro-conductive textile panel 18, a carbon based anti-bacterial cover 20, and a simple peelable backing or cover 22 which may be released from an adhesive layer applied over the outer surface of either the layer 16 or layer 20.

1: A heated wound dressing consisting of an electroconductive textile and control circuitry for controlling said electroconductive textile, characterised in that the textile is place directly in contact with the wound to be treated.

2: A dressing according to claim 1 characterised in that the textile is fibre, yarn or other fabric which is carbonised to enable use as a resistance heater element.

3: A dressing according to claim 2 characterised in that the Element is woven or knitted.

4: A dressing according to claim 3 characterised in that the element is provided with electrical terminals and is powered from a suitable low voltage source.

5: A dressing according to claim 1 characterised in that the dressing further includes an insulating layer to reduce energy requirements and ensure significant heat is not lost to atmosphere.

6: A dressing according to claim 1 characterised in that control circuitry is provided which relies on a feedback temperature measurement to ensure uniform and stable temperature is maintained.

7: A dressing according to claim 6 characterised in that the feedback temperature measurement is effected by a thermistor.

8: A dressing according to claim 6 characterised in that the control circuitry includes switching components and/or software control to ensure that the element is only used over a “safe” life span.

9: A dressing according to claim 8 characterised in that the control circuitry incorporates one or more of the following features:
Multi channel operation, probably 5 in total to correspond with anatomic areas;
Precise temperature control for prolonged or indefinite periods of time;
Virtually no operational temperature hysterisis;
Reciprocal temperature feedback from the patient's skin tissue;
Over temperature failsafe cut off;
Re-programmability of operational parameters.

10: A dressing according to claim 8 characterised in that the software for controlling the operation of the control circuitry (i. e. front end software for initially programming the solid state devices on the control circuitry) include one or more of the following features:
Ability to set target temperature for 5 individual dressings;
Ability to display such temperatures in real time;
Ability to change such temperatures;
To have feedback from the control module that system function continued;
To be warned if system or power failure had taken place;
To re-initiate system operation without the need to re-boot;
Access a menu of pre-set symmetrical temperature and load a chosen pre-set to control module;
To receive confirmation that the pre-set had been accepted;
To have new pre-set settings automatically updated on display.

11: A dressing according to claim 1 which includes one or more of the following features:
Breathable and waterproof“Throw away”system where element is an integral part of the dressing;
Breathable and waterproof“Throw away”element where element is separate to the dressing;
Breathable porous“Throw away”system where element is an integral part of the dressing;
Breathable porous“Throw away”element where element is separate to the dressing;
Impermeable“Throw away”system where element is an integral part of the dressing;
Impermeable“Throw away”element where element is separate to the dressing.

12: A dressing according to claim 1 characterised in that the electro-conductive textile is laminated to a dressing material.

13: A dressing according to any of claim 1 characterised in that the electro-conductive textile heating element is embodied in the dressing in one of the following ways:
unlaminated and separate from the dressing material;
heating element laminated but breathable (unsealed) and separate to the dressing material. Both heating element and dressing to be one use throw away system;
heating element sealed into an envelope that could be laundered or sterilised (breathable or non-breathable) and separate to the dressing material. The dressing would be throw away but the element could be removed from the envelope and the envelope discarded or sterilised;
heating element sealed into awashable/sterilisable envelope (breathable or non-breathable) and separate to the dressing material;
heating element sealed into an impermeablewashable/sterilisable laminate that would be separate to the underlying dressing and would have the thermally insulating material laminated directly to its upper surface and/or as a separate layer positioned above the heating element;
heating element sealed into an impermeable laminate that would be an integral part of the underlying dressing and would have the thermally insulating material laminated directly to its upper surface and/or as a separate layer positioned above the heating element.

14: A dressing according to claim 13 wherein the dressing is provided in an initially sealed and sterilised package from which it is removed immediately prior to use, said dressing further having a release paper adhered to the surface which in use is directly applied to the wound, such that on removing the release paper from the dressing, the dressing is adhered directly to the skin.

15: A dressing according to claim 13 characterised in that the electro-conductive textile heating element has laminated thereto a neoprene lamination to each face of the element.

16: A dressing according to claim 15 characterised in that the lower face of the heating element which is most proximate the patient is covered with a very thin, typically 1 mm layer of thermally conductive neoprene whereas the upper surface of said heating element is thermally efficient, typically 3mm thick neoprene layer, optionally having a silver Titanium reflective backing to it.

17: A heated wound dressing consisting of an electro-conductive textile and control circuitry for controlling said electroconductive textile, characterised in that the textile is sealingly encapsulated within an envelope type package.

18: A dressing according to claim 17 characterised in that the textile is fibre, yarn or other fabric which is carbonised to enable use as a resistance heater element.

19: A dressing according to claim 18 characterised in that the Element is woven or knitted.

20: A dressing according to any of claim 17 characterised in that the element is provided with electrical terminals and is powered from a suitable low voltage source.

21: A dressing according to any of claim 17 characterised in that the dressing further includes an insulating layer to reduce energy requirements and ensure significant heat is not lost to atmosphere.

22: A dressing according to any of claim 17 characterised in that control circuitry is provided which relies on a feedback temperature measurement to ensure uniform and stable temperature is maintained.

23: A dressing according to claim 22 characterised in that the feedback temperature measurement is effected by a thermistor.

24: A dressing according to claim 22 characterised in that the control circuitry includes switching components and/or software control to ensure that the element is only used over a“safe”life span.

25: A dressing according to claim 24 characterised in that the control circuitry incorporates one or more of the following features:
Multi channel operation, probably 5 in total to correspond with anatomic areas;
Precise temperature control for prolonged or indefinite periods of time;
Virtually no operational temperature hysterisis;
Reciprocal temperature feedback from the patient's skin tissue;
Over temperaturefailsafe cut off;
Re-programmability of operational parameters.

26: A dressing according to claim 24 characterised in that the software for controlling the operation of the control circuitry (i. e. front end software for initially programming the solid state devices on the control circuitry) include one or more of the following features:
Ability to set target temperature for 5 individual dressings;
Ability to display such temperatures in real time;
Ability to change such temperatures;
To have feedback from the control module that system function continued;
To be warned if system or power failure had taken place;
To re-initiate system operation without the need to re-boot;
Access a menu of pre-set symmetrical temperature and load a chosen pre-set to control module;
To receive confirmation that the pre-set had been accepted;
To have new pre-set settings automatically updated on display.

27: A dressing according to any of claim 17 which includes one or more of the following features:
Breathable and waterproof“Throwaway”system where element is an integral part of the dressing;
Breathable and waterproof“Throw away”element where element is separate to the dressing;
Breathable porous“Throw away”system where element is an integral part of the dressing;
Breathable porous“Throw away”element where element is separate to the dressing;
Impermeable“Throw away”system where element is an integral part of the dressing;
Impermeable“Throw away”element where element is separate to the dressing.

28: A dressing according to any of claim 17 characterised in that the electro-conductive textile is laminated to a dressing material.

29: A dressing according to any of claim 17 characterised in that the electro-conductive textile heating element is embodied in the dressing in one of the following ways:
unlaminated and separate from the dressing material;
heating element laminated but breathable (unsealed) and separate to the dressing material. Both heating element and dressing to be one use throw away system;
heating element sealed into an envelope that could be laundered or sterilised (breathable or non-breathable) and separate to the dressing material. The dressing would be throw away but the element could be removed from the envelope and the envelope discarded or sterilised;
heating element sealed into awashable/sterilisable envelope (breathable or non-breathable) and separate to the dressing material;
heating element sealed into an impermeable washable/sterilisable laminate that would be separate to the underlying dressing and would have the thermally insulating material laminated directly to its upper surface and/or as a separate layer positioned above the heating element.
heating element sealed into an impermeable laminate that would be an integral part of the underlying dressing and would have the thermally insulating material laminated directly to its upper surface and/or as a separate layer positioned above the heating element.

30: A dressing according to any of claim 17 wherein the dressing is provided in an initially sealed and sterilised package from which it is removed immediately prior to use, said dressing further having a release paper adhered to the surface which in use is directly applied to the wound, such that on removing the release paper from the dressing, the dressing is adhered directly to the skin.

31: A dressing according to any of claim 17 characterised in that the electro-conductive textile heating element has laminated thereto a neoprene lamination to each face of the element.

32: A dressing according to claim 31 characterised in that the lower face of the heating element which is most proximate the patient is covered with a very thin, typically 1 mm layer of thermally conductive neoprene whereas the upper surface of said heating element is thermally efficient, typically 3 mm thick neoprene layer, optionally having a silver Titanium reflective backing to it.
